(19) United States
(12) Patent Application Publication
(10) Pub. No.: US 2024/0252415 A1
(43) Pub. Date: Aug. 1, 2024

(54) SKIN CARE METHODS AND FORMULATIONS

(71) Applicant: Cybele Microbiome, Inc., San Diego, CA (US)

(72) Inventors: Nicole M. Scott, San Diego, CA (US); Mannige Vikram Rao, Chapel Hill, NC (US); James Lamoureux, San Diego, CA (US)

(21) Appl. No.: 18/562,497

(22) PCT Filed: May 20, 2022
(86) PCT No.: PCT/US2022/030315

§ 371 Date: Nov. 20, 2023

Related U.S. Application Data
(60) Provisional application No. 63/191,230, filed on May 20, 2021.

(51) Int. Cl. A61K 8/68; A61Q 19/00; A61Q 19/08

(52) U.S. Cl. CPC A61K 8/68; A61Q 19/005; A61Q 19/007; A61Q 19/08; A61K 2800/78

(57) ABSTRACT

Described herein are formulations that comprise at least about 50% by weight water and no greater than about 1% by weight of a sphingolipid. The formulations can be applied to a region of skin of a subject. After the formulation is applied to the region of the skin of the subject, a biochemical pathway can be activated by the formulation to cause the production of a ceramide on the region of the skin of the subject. The amount of the ceramide produced on the region of the skin of the subject can be greater than a baseline amount of the ceramide that is produced in the absence of the formulation. In various examples, the increased amount of ceramide produced after applying the formulation to the region of the skin of the subject can modify a condition present on the region of the skin of the subject.

This application claims priority to U.S. Provisional Patent Application Ser. No. 63/191,230 filed May 20, 2021, and entitled “Skin Care Methods and Formulations”, the entire contents of which is incorporated by reference herein in its entirety. In addition, the contents of US Provisional Patent Application Ser. No. 63/181,821 filed on Apr. 29, 2021, and entitled “Skin Care Methods and Formulations” and the contents of PCT application PCT/US2022/027148 filed on Apr. 29, 2022, and entitled “Analyzing Genomics Data and Analytical Data” are both incorporated by reference herein in their entirety.

### BACKGROUND

The skin of mammals can support many microorganisms including bacteria, protists, archaea, fungi, and viruses. The microorganisms supported by the skin of mammals can comprise a skin microbiota. A combination of the microorganisms living on the skin and the genetic material of the microorganisms can comprise a skin microbiome. The microbiota can be different on different locations of the skin of the same subject. Additionally, the skin microbiota can vary across individual subjects. Skin health for a particular subject can be based, at least partly, on the skin microbiota of the subject.

### BRIEF DESCRIPTION OF THE DRAWINGS

The present disclosure is illustrated by way of example and not limitation in the figures of the accompanying drawings, in which like references indicate similar elements.

FIG. 1 is a diagram illustrating an example method to apply a formulation including at least about 50% by weight water and no greater than about 1% by weight of a sphingolipid to a region of skin of a subject, in accordance with one or more example implementations.

FIG. 2A shows a picture of a first subject in study #1 prior to use of a formulation described herein and FIG. 2B shows a picture of the first subject after use of the formulation.

FIG. 3A shows a picture of a second subject in study #1 prior to use of a formulation described herein and FIG. 3B shows a picture of the second subject after use of the formulation.

FIG. 4A shows a picture of a third subject in study #1 prior to use of a formulation described herein and FIG. 4B shows a picture of the third subject after use of the formulation.

FIG. 5A shows a picture of a fourth subject in study #1 prior to use of a formulation described herein and FIG. 5B shows a picture of the fourth subject after use of the formulation.

FIG. 6A shows a picture of a fifth subject in study #1 prior to use of a formulation described herein and FIG. 6B shows a picture of the fifth subject after use of the formulation.

FIG. 7A shows a picture of a first subject that participated in study #2 prior to use of a formulation described herein and FIG. 7B shows a picture of the first subject after use of the formulation.

FIG. 8A shows a picture of a second subject that participated in study #2 prior to use of a formulation described herein and FIG. 8B shows a picture of the second subject after use of the formulation.

FIG. 9A shows a picture of a third subject that participated in study #2 prior to use of a formulation described herein and FIG. 9B shows a picture of the third subject after use of the formulation.

FIG. 10A shows a picture of a fourth subject that participated in study #2 prior to use of a formulation described herein and FIG. 10B shows a picture of the fourth subject after use of the formulation.

FIG. 11A shows a picture of a fifth subject that participated in study #2 prior to use of a formulation described herein and FIG. 11B shows a picture of the fifth subject after use of the formulation.

FIG. 12A shows a picture of a sixth subject that participated in study #2 prior to use of a formulation described herein and FIG. 12B shows a picture of the sixth subject after use of the formulation.

FIG. 13A shows a picture of an additional subject having an upper wound and a lower scar prior to use of a formulation described herein and FIG. 13B shows a picture of the additional subject after use of the formulation.

FIG. 14 shows normalized measures of abundance of a number of bacteria for subjects of study #2 characterized with normal skin before and after treatment with the formulation.

FIG. 15 shows normalized measures of abundance of a number of bacteria for subjects of study #2 characterized with sensitive skin before and after treatment with the formulation.

### DETAILED DESCRIPTION

Typically, cosmetics and treatments applied to the skin include active amounts of ingredients to treat a skin condition or to improve the health of the skin of individuals. In implementations described herein, formulations are applied to the skin of individuals that include prebiotics that cause biochemical pathways of individuals to be activated to produce compounds that are used in the treatment of skin conditions and improving the health of the skin of individuals.

Described herein are formulations that comprise at least about 50% by weight water and no greater than about 1% by weight of a sphingolipid. The formulations can be applied to a region of skin of a subject. After the formulation is applied to the region of the skin of the subject, a biochemical pathway can be activated by the formulation to cause the production of a ceramide on the region of the skin of the subject. The amount of the ceramide produced on the region of the skin of the subject can be greater than a baseline amount of the ceramide that is produced in the absence of the formulation. In various examples, the increased amount of ceramide produced after applying the formulation to the region of the skin of the subject can modify a condition present on the region of the skin of the subject. To illustrate, as the amount of the ceramide present on the region of the skin of the subject increases beyond a baseline amount of the ceramide as a result of applying the formulation to the region of the skin of the subject, a condition present on the region of the skin of the subject can be modified. In one or more examples, the condition present on the region of the skin of the individual can improve over a period of time that the formulation is applied to the region of the skin of the subject. In this way, the formulation can include a prebiotic that causes a biochemical pathway to be activated to generate a postbiotic that includes at least one ceramide. Further, the formulation does not inhibit the skin microbiota or affect the viability of the microbes present in the microbiota of the subject. Shifting microbe communities can increase negative and pathogenic organisms that can reduce the protective skin barrier and reduce skin health. The formulations described herein are microbiome safe and safe for the subjects to which the formulations can be applied. In various examples, the formulations described herein can improve the composition of bacteria and the amounts of bacteria present on the skin of the individual.

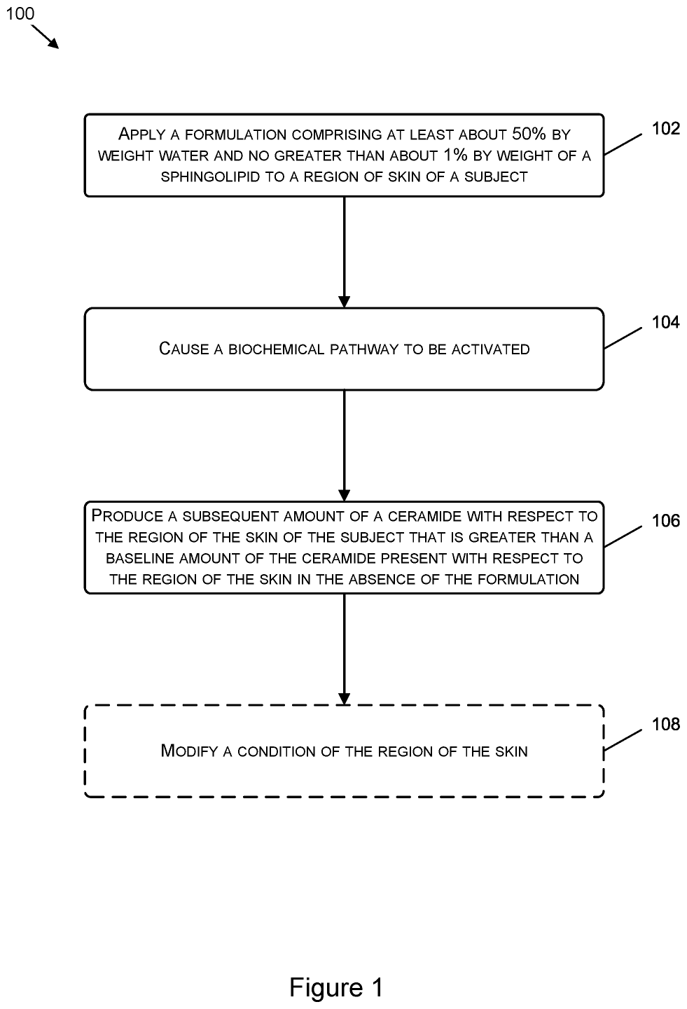

FIG. 1 is a diagram illustrating an example method 100 to apply a formulation including at least about 50% by weight water and no greater than about 1% by weight of a sphingolipid to a region of skin of a subject, in accordance with one or more example implementations. The method can include, at 102, applying a formulation comprising at least about 50% by weight water and no greater than about 1% by weight of a sphingolipid to a region of skin of a subject. The subject can include a mammal. In various examples, the subject can include a human. In one or more additional examples, the subject can include a mammal that is different from a human, such as at least one of a horse, a dog, a cow, a pig, or a mouse.

In various examples, the formulation can comprise at least about 40% by weight water, at least 42% by weight water, at least about 45% by weight water, at least about 48% by weight water, at least about 50% by weight water, at least about 52% by weight water, or at least about 55% by weight water. In addition, the formulation can include no greater than about 70% by weight water, no greater than about 68% by weight water, no greater than about 65% by weight water, no greater than about 62% by weight water, or no greater than about 60% by weight water. In one or more illustrative examples, the formulation can include from about 40% by weight water to about 70% by weight water, from about 50% by weight water to about 65% by weight water, or from about 52% by weight water to about 60% by weight water.

In one or more examples, the sphingolipid can comprise sphingosine. The formulation can include at least about 0.0005% by weight sphingosine, at least about 0.0008% by weight sphingosine, at least about 0.0010% by weight sphingosine, at least about 0.0012% by weight sphingosine, at least about 0.0015% by weight sphingosine, at least about 0.0018% by weight sphingosine, or at least about 0.002% by weight sphingosine. Additionally, the formulation can include no greater than about 0.005% by weight sphingosine, no greater than about 0.008% by weight sphingosine, no greater than about 0.010% by weight sphingosine, no greater than about 0.015% by weight sphingosine, no greater than about 0.020% by weight sphingosine, no greater than about 0.030% by weight sphingosine, or no greater than about 0.050% by weight sphingosine. In one or more illustrative examples, the formulation can include about 0.0005% by weight sphingosine to about 0.005% by weight sphingosine, from about 0.0005% by weight sphingosine to about 0.05% by weight sphingosine, or from about 0.0010% by weight sphingosine to about 0.005% by weight sphingosine.

The formulation can also include one or more diols. For example, the formulation can include one or more diols having from 3 to 6 carbon atoms. In one or more examples, the formulation can include at least two diols. In various examples, the formulation can include from about 20% by weight to about 40% by weight of a first diol having from 3 to 6 carbon atoms and from about 2% by weight to about 10% by weight of a second diol having from about 3 to 6 carbon atoms. In one or more examples, the formulation can include 1,3-butanediol. In one or more additional examples, the formulation can include 1,2-propanediol. In one or more illustrative examples, the formulation can include 1,3-butanediol and 1,2-propanediol.

The formulation can include at least about 10% by weight 1,3-butanediol, at least about 12% by weight 1,3-butanediol, at least about 15% by weight 1,3-butanediol, at least about 18% by weight 1,3-butanediol, at least about 20% by weight 1,3-butanediol, at least about 22% by weight by weight 1,3-butanediol, or at least about 25% by weight 1,3-butanediol. The formulation can also include no greater than about 40% by weight 1,3-butanediol, no greater than about 38% by weight 1,3-butanediol, no greater than about 35% by weight 1,3-butanediol, no greater than about 32% by weight 1,3-butanediol, or no greater than about 30% by weight 1,3-butanediol. In one or more illustrative examples, the formulation can include from about 15% by weight 1,3-butanediol to about 30% by weight 1,3-butanediol, from about 20% by weight to about 30% by weight 1,3-butanediol, or from about 22% by weight 1,3-butanediol to about 28% by weight 1,3-butanediol.

Additionally, the formulation can include at least about 1% by weight 1,2-propanediol, at least about 2% by weight 1,2-propanediol, at least about 3% by weight 1,2-propanediol, at least about 4% by weight 1,2-propanediol, or at least about 5% by weight 1,2-propanediol. The formulation can also include no greater than about 15% by weight 1,2-propanediol, no greater than about 12% by weight 1,2-propanediol, no greater than about 10% by weight 1,2-propanediol, or no greater than about 8% by weight 1,2-propanediol. In one or more illustrative examples, the formulation can include from about 1% by weight 1,2-propanediol to about 15% by weight 1,2-propanediol, from about 2% by weight 1,2-propanediol to about 10% by weight 1,2-propanediol, or from about 2% by weight 1,2-propanediol to about 8% by weight 1,2-propanediol. In one or more illustrative examples, 1,2-propanediol may be absent from the formulation.

Further, the formulation can include an amount of glycerin. In various examples, the formulation can include at least about 2% by weight glycerin, at least about 3% by weight glycerin, at least about 5% by weight glycerin, at least about 7% by weight glycerin, or at least about 9% by weight glycerin. The formulation can also include no greater than about 20% by weight glycerin, no greater than about 18% by weight glycerin, no greater than about 15% by weight glycerin, or no greater than about 12% by weight glycerin. In one or more illustrative examples, the formulation can include from about 2% by weight glycerin to about 20% by weight glycerin, from about 3% by weight glycerin to about 15% by weight glycerin, or from about 7% by weight glycerin to about 12% by weight glycerin.

The formulation can include an amount of squalene. For example, the formulation can include at least about 0.1% by weight squalene, at least about 0.3% by weight squalene, at least about 0.5% by weight squalene, at least about 0.7% by weight squalene, or at least about 1.0% by weight squalene. In addition, the formulation can include no greater than about 3% by weight squalene, no greater than about 2.5% by weight squalene, no greater than about 2.0% by weight squalene, no greater than about 1.5% by weight squalene, or no greater than about 1.2% by weight squalene. In one or more illustrative examples, the formulation can include from about 0.1% by weight squalene to about 3% by weight squalene, from about 0.5% by weight squalene to about 2.0% by weight squalene, or from about 0.7% by weight squalene to about 1.2% by weight squalene.

The formulation can also include an amount of isohexadecane. To illustrate, the formulation can include at least about 0.05% by weight isohexadecane, at least about 0.08% by weight isohexadecane, at least about 0.10% by weight isohexadecane, at least about 0.15% by weight isohexadecane, at least about 0.20% by weight isohexadecane, at least about 0.22% by weight isohexadecane, or at least about 0.25% by weight isohexadecane. The formulation can also include no greater than about 0.50% by weight isohexadecane, no greater than about 0.45% by weight isohexadecane, no greater than about 0.40% by weight isohexadecane, no greater than about 0.35% by weight isohexadecane, or no greater than about 0.30% by weight isohexadecane. In one or more illustrative examples, the formulation can include from about 0.05% by weight isohexadecane to about 0.50% by weight isohexadecane, from about 0.10% by weight isohexadecane to about 0.40% by weight isohexadecane, or from about 0.20% by weight isohexadecane to about 0.30% by weight isohexadecane.

In addition, the formulation can include an amount of palmitic acid. In one or more examples, the formulation can include at least about 0.0005% by weight palmitic acid, at least about 0.0008% by weight palmitic acid, at least about 0.0010% by weight palmitic acid, at leas about 0.0012% by weight palmitic acid, at least about 0.0015% by weight palmitic acid, at least about 0.0018% by weight palmitic acid, or at least about 0.0020% by weight palmitic acid. The formulation can also include no greater than about 0.0050% by weight palmitic acid, no greater than about 0.0045% by weight palmitic acid, no greater than about 0.0040% by weight palmitic acid, no greater than about 0.0035% by weight palmitic acid, no greater than about 0.0030% by weight palmitic acid, no greater than about 0.0025% by weight palmitic acid. In one or more illustrative examples, the formulation can include from about 0.0005% by weight palmitic acid to about 0.0050% by weight palmitic acid, from about 0.0010% by weight palmitic acid to about 0.0040% by weight palmitic acid, or from about 0.0015% by weight palmitic acid to about 0.0025% by weight palmitic acid.

The formulation can include an amount of polysorbate 80. To illustrate, the formulation can include at least about 0.04% by weight polysorbate 80, at least about 0.05% by weight polysorbate 80, at least about 0.06% by weight polysorbate 80, at least about 0.07% by weight polysorbate 80, or at least about 0.08% by weight polysorbate 80. The formulation can also include no greater than about 0.20% by weight polysorbate 80, no greater than about 0.18% by weight polysorbate 80, no greater than bout 0.15% by weight polysorbate 80, no greater than about 0.12% by weight polysorbate 80, or no greater than about 0.10% by weight polysorbate 80. In one or more illustrative examples, the formulation can include from about 0.04% by weight polysorbate 80 to about 0.20% by weight polysorbate 80, from about 0.06% by weight polysorbate 80 to about 0.15% by weight polysorbate 80, or from about 0.08% by weight polysorbate 80 to about 0.10% by weight polysorbate 80.

The formulation can further include an amount of a sodium acrylate/sodium acryloyldimethyl taurate copolymer. For example, the formulation can include at least about 0.05% by weigh sodium acrylate/sodium acryloyldimethyl taurate copolymer, at least about 0.10% by weight sodium acrylate/sodium acryloyldimethyl taurate copolymer, at least about 0.15% by weight sodium acrylate/sodium acryloyldimethyl taurate copolymer, at least about 0.20% by weight sodium acrylate/sodium acryloyldimethyl taurate copolymer, at least about 0.25% by weight sodium acrylate/sodium acryloyldimethyl taurate copolymer, at least about 0.30% by weight sodium acrylate/sodium acryloyldimethyl taurate copolymer, at least about 0.35% by weight sodium acrylate/sodium acryloyldimethyl taurate copolymer, or at least about 0.40% by weight sodium acrylate/sodium acryloyldimethyl taurate copolymer. Further, the formulation can include no greater than about 1.5% by weight sodium acrylate/sodium acryloyldimethyl taurate copolymer, no greater than about 1.2% by weight sodium acrylate/sodium acryloyldimethyl taurate copolymer, no greater than about 1.0% by weight sodium acrylate/sodium acryloyldimethyl taurate copolymer, no greater than about 0.8% by weight sodium acrylate/sodium acryloyldimethyl taurate copolymer, or no greater than about 0.5% by weight sodium acrylate/sodium acryloyldimethyl taurate copolymer. In one or more illustrative examples, the formulation can include from about 0.10% by weight sodium acrylate/sodium acryloyldimethyl taurate copolymer to about 1.5% by weight sodium acrylate/sodium acryloyldimethyl taurate copolymer, from about 0.20% by weight sodium acrylate/sodium acryloyldimethyl taurate copolymer to about 1.0% by weight sodium acrylate/sodium acryloyldimethyl taurate copolymer, or from about 0.40% by weight sodium acrylate/sodium acryloyldimethyl taurate copolymer to about 0.50% by weight sodium acrylate/sodium acryloyldimethyl taurate copolymer.

In one or more illustrative examples, the formulation can include an amount of water, an amount of a sphingolipid, and one or more carrier compounds, such as at least one of one or more conditioning agents, one or more cleansing agents, one or more emulsifying agents, or one or more emulsion stabilizers. The one or more carrier compounds can provide a cosmetically pleasing delivery system for the prebiotics. Factors that affect the selection of the carrier compounds included in the formulation can include hydrophobicity, pH, solubility, and long-term stability to maintain formulation efficacy. The one or more carrier compounds are selected to minimize any impact of the formulation on health of the microbiota of the subject. In one or more examples, the cleansing agent can include palmitic acid. The one or more conditioning agents can include at least one of 1,3 butanediol, isohexadecane, or squalene. In various examples, the emulsifying agent can include polysorbate 80. The emulsion stabilizer can include a sodium acrylate and sodium acryloyldimethyl taurate copolymer.

In one or more examples, the formulation can include from about 50% by weight water to about 65% by weight water, from about 20% by weight 1,3-butanediol to about 30% by weight 1,3-butanediol, from about 5% by weight glycerin to about 15% by weight glycerin, from about 2% by weight 1,2-propanediol to about 10% by weight 1,2-propanediol, and from about 0.0005% by weight sphingosine to about 0.005% by weight sphingosine. The formulation can also include from about 0.7% by weight squalene to about 1.2% by weight squalene, from about 0.20% by weight isohexadecane to about 0.30% by weight isohexadecane, from about 0.0015% by weight palmitic acid to about 0.0025% by weight palmitic acid, from about 0.08% by weight polysorbate 80 to about 0.10% by weight polysorbate 80, and from about 0.40% by weight sodium acrylate/sodium acryloyldimethyl taurate copolymer to about 0.50% by weight sodium acrylate/sodium acryloyldimethyl taurate copolymer.

The formulation can be made by combining an amount of water, an amount of glycerin, an amount of one or more diols, an amount of one or more conditioning agents, an amount of one or more emulsifying agents, an amount of one or more emulsifying stabilizers, an amount of one or more cleansing agents, and an amount of sphingosine. In one or more examples, an amount of sphingosine is heated and combined with an amount of squalene to produce a mixture of sphingosine and squalene. In one or more illustrative examples, the mixture of sphingosine and squalene can be mixed with water, 1,3-butanediol, 1,2-propanediol, glycerin, palmitic acid, isohexadecane, polysorbate 80, and a sodium acrylate and sodium acryloyldimethyl taurate copolymer to produce the formulation.

Additionally, the method 100 can include, at 104, causing a biochemical pathway to be activated. The biochemical pathway can include a biochemical pathway that includes a sphingolipid as an intermediate component. In one or more examples, the biochemical pathway can include the sphingosine-1-phosphate biochemical pathway.

Further, at 106, the method 100 can include producing a subsequent amount of a ceramide with respect to the region of the skin of the subject that is greater than a baseline amount of the ceramide present with respect to the region of the skin in the absence of the formulation. In one or more examples, the subsequent amount of the ceramide produced can be at least about 2% greater than the baseline amount of the ceramide, at least about 5% greater than the baseline amount of the ceramide, at least about 10% greater than the baseline amount of the ceramide, at least about 15% greater than the baseline amount of the ceramide, at least about 20% greater than the baseline amount of the ceramide, at least about 25% greater than the baseline amount of the ceramide, at least about 30% greater than the baseline amount of the ceramide, at least about 40% greater than the baseline amount of the ceramide, at least about 50% greater than the baseline amount of the ceramide, at least about 100% greater than the baseline amount of the ceramide, or at least about 200% greater than the baseline amount of the ceramide.

The method 100 can also include, at 108, modifying a condition of the region of the skin. In various examples, the subsequent amount of ceramide produced by applying the formulation to the region of the skin of the individual can modify the condition of the region of the skin. In one or more illustrative examples, the condition can comprise at least one of dermatitis, erythema, precancerous lesions, mottling, scaling, dark spots, scars, redness, lines, or wrinkles. The subsequent amount of ceramide can be functional in that the subsequent amount of ceramide produces changes to the skin of the subject that would not be produced by the baseline amount of the ceramide. In at least some examples, the subsequent amount of ceramide can produce changes to the skin of the subject that would not be produced by the baseline amount of the ceramide over a given period of time. The formulation can modify a condition of the region of the skin of the subject after being applied to the region of the skin of the subject at least once within individual consecutive 48-hour periods for at least 3 consecutive 48-hour periods. Further, the formulation can modify a condition of the region of the skin of the subject after being applied to the region of the skin of the subject at least once per day for at least 4 consecutive days.

In one or more examples, the subsequent amount of the ceramide is present on the region of the skin of the subject for at least 10 hours after the formulation is applied to the region of the skin of the subject. In addition, the subsequent amount of the ceramide is present on the region of the skin of the subject from about 0.5 hours to about 24 hours after the formulation is applied to the region of the skin of the subject.

In various examples, a dose of the formulation can include at least 0.01 milliliters (mL), at least 0.02 mL, at least 0.05 mL, at least 0.08 mL, at least 0.10 mL, at least 0.12 mL, at least 0.15 mL, at least 0.18 mL, at least 0.20 mL, at least 0.22 mL, at least 0.25 mL, at least 0.28 mL, at least 0.30 mL, at least 0.32 mL, at least 0.35 mL, at least 0.38 mL, or at least 0.40 mL. In one or more examples, a dose of the formulation can be from 0.01 mL to 2 mL, from 0.05 mL to 1 mL, from 0.10 mL to 0.80 mL, from 0.20 mL to 0.50 mL, from 0.30 mL to 0.60 mL, or from 0.30 mL to 0.50 mL. In one or more illustrative examples, an effective amount of the formulation can include at least 1 dose, at least 2 doses, at least 3 doses, at least 5 doses, at least 8 doses, at least 10 doses, at least 12 doses, at least 15 doses, at least 18 doses, or at least 20 doses. In one or more additional examples, an effective amount of the formulation can be from 1 dose to 200 doses, from 1 dose to 150 doses, from 1 dose to 100 doses, from 1 dose to 75 doses, from 1 dose to 50 doses, from 1 does to 40 doses, from 1 dose to 30 doses, from 1 dose to 20 doses, from 1 dose to 10 doses, from 3 doses to 200 doses, from 3 doses to 150 doses, from 3 doses to 100 doses, from 3 doses to 75 doses, from 3 doses to 50 doses, from 3 doses to 40 doses, from 3 doses to 30 doses, from 3 doses to 20 doses, from 3 doses to 10 doses, from 5 doses to 200 doses, from 5 doses to 150 doses, from 5 doses to 100 doses, from 5 doses to 75 doses, from 5 doses to 50 doses, from 5 doses to 40 doses, from 5 doses to 30 doses, from 5 doses to 20 doses, from 5 doses to 10 doses, from 6 doses to 200 doses, from 6 doses to 150 doses, from 6 doses to 100 doses, from 6 doses to 75 doses, from 6 doses to 50 doses, from 6 doses to 40 doses, from 6 doses to 30 doses, from 6 doses to 20 doses, from 6 doses to 10 doses, from 8 doses to 200 doses, from 8 doses to 150 doses, from 8 doses to 100 doses, from 8 doses to 75 doses, from 8 doses to 50 doses, from 8 doses to 40 doses, from 8 doses to 30 doses, from 8 doses to 20 doses, from 8 doses to 10 doses, from 10 doses to 200 doses, from 10 doses to 150 doses, from 10 doses to 100 doses, from 10 doses to 75 doses, from 10 doses to 50 doses, from 10 doses to 40 doses, from 10 doses to 30 doses, or from 10 doses to 20 doses.

In various examples, the formulation can be applied to a region of skin in effective amounts once per day, twice per day, three times per day, four times per day, or five times per day. The formulation can also be applied to a region of skin to treat a skin condition for at least one day, at least 2 days, at least 3 days, at least 4 days, at least 5 days, at least 6 days, at least 7 days, at least 8 days, at least 9 days, at least 10 days, at least 15 days, at least 25 days, at least 30 days, at least 40 days, at least 50 days, at least 75 days, at least 100 days, at least 150 days, at least 200 days, at least 250 days, at least 300 days, at least 350 days. In various examples, the formulation can be applied to skin to treat a condition until the condition is no longer present on the skin.

In one or more illustrative examples, the formulation can be applied to skin of an individual to treat one or more conditions. For example, the formulation can be applied to the skin of an individual to treat atopic dermatitis. In addition, the formulation can be applied to the skin of an individual to treat melanoma. Further, the formulation can be applied to the skin of an individual to treat redness. Also, the formulation can be applied to the skin of an individual to treat dryness. In one or more examples, the formulation can be applied to the skin of an individual to treat scarring. In one or more additional examples, the formulation can be applied to the skin of an individual to treat precancerous lesions. In one or more further examples, the formulation can be applied to the skin of an individual to treat acne. In still other examples, the formulation can be applied to the skin of an individual to treat wounds, such as cuts, sores, bites, or lesions. Additionally, the formulation can be applied to the skin of an individual to treat wrinkles. In various examples, the formulation can be applied to the skin of an individual to treat hyperpigmentation. The formulation can also be applied to the skin of an individual to treat dark spots. In still further examples, the formulation can be applied to the skin of an individual to treat erythema. In still additional examples, the formulation can be applied to the skin of an individual to treat unevenness of skin color. In one or more examples, the formulation can be applied to the skin of an individual to treat flakiness. In one or more additional examples, the formulation can be applied to the skin of an individual to treat scaliness. In one or more further examples, the formulation can be applied to the skin of an individual to treat comedones.

The formulation can also be applied to the skin of an individual to increase moisture of the skin included in the region being treated. In addition, the formulation can be applied to the skin of an individual to reduce redness of the skin included in the region being treated. Further, the formulation can be applied to the skin of an individual to decrease inflammation of the skin included in the region being treated. In various examples, the formulation can be applied to the skin of an individual to decrease sensitivity of the skin included in the region being treated. In one or more illustrative examples, a skin condition can begin to improve in response to applying from 0.15 mL to 0.50 mL of the formulation to a region of the skin for at least three days. In one or more examples, the formulation can be applied to the region of the skin of the individual two times per day. In various examples, a dose of the formulation can be dispensed as a spray from a container that includes a volume of the formulation.

The formulation can be applied to a number of parts of the body of an individual. For example, the formulation can be applied to a face of an individual. In addition, the formulation can be applied to a neck of an individual. Further, the formulation can be applied to at least a portion of at least one of a limb or appendage of an individual. The formulation can also be applied to at least a portion of at least one of a torso, back, or stomach of an individual. In one or more examples, the formulation can be applied to at least a portion of a scalp of an individual.

A numbered non-limiting list of aspects of the present subject matter is presented below.

Aspect 1. A formulation comprising: from about 50% by weight to about 65% by weight water; from about 20% by weight to about 30% by weight 1,3-butanediol; from about 5% by weight to about 15% by weight glycerin; from about 2% by weight to about 10% by weight 1,2-propanediol; and from about 0.0005% by weight to about 0.005% by weight sphingosine.

Aspect 2. The formulation of aspect 1, comprising: from about 0.7% by weight squalene to about 1.2% by weight squalene and from about 0.0015% by weight palmitic acid to about 0.0025% by weight palmitic acid.

Aspect 3. A formulation comprising: at least about 50% by weight water; and no greater than about 1% by weight of a sphingolipid.

Aspect 4. The formulation of aspect 3, wherein the sphingolipid comprises sphingosine.

Aspect 5. The formulation of aspect 3 or 4, comprising from about 20% by weight to about 40% by weight of one or more diols having from 3 to 6 carbon atoms.

Aspect 6. The formulation of aspect 5, comprising from about 20% by weight to about 40% by weight of a first diol having from 3 to 6 carbon atoms and from about 2% by weight to about 10% by weight of a second diol having from about 3 to 6 carbon atoms.

Aspect 7. The formulation of aspect 6, comprising from about 20% by weight to about 30% by weight of 1,3-butanediol and from about 2% by weight to about 10% by weight of 1,2-propanediol.

Aspect 8. The formulation of any one of aspects 3 to 7, comprising from about 7% by weight to about 12% by weight glycerin.

Aspect 9. The formulation of any one of aspects 3 to 8, comprising one or more conditioning agents.

Aspect 10. The formulation of aspect 9, wherein the one or more conditioning agents include at least one of 1,3 butanediol, isohexadecane, or squalene.

Aspect 11. The formulation of aspect 9 or 10, comprising no greater than about 0.5% by weight isohexadecane.

Aspect 12. The formulation of any one of aspects 9 to 11, comprising from about 0.10% by weight to about 0.40% by weight isohexadecane.

Aspect 13. The formulation of any one of aspects 9 to 12, comprising no greater than about 2% by weight squalene.

Aspect 14. The formulation of aspect 13, comprising from about 0.7% by weight to about 1.2% by weight squalene.

Aspect 15. The formulation of any one of aspects 3 to 14, comprising an emulsifying agent.

Aspect 16. The formulation of aspect 15, comprising no greater than about 0.2% by weight polysorbate 80.

Aspect 17. The formulation of aspect 15 or 16, comprising from about 0.06% by weight to about 0.15% by weight polysorbate 80.

Aspect 18. The formulation of any one of aspects 3 to 17, comprising an emulsion stabilizer.

Aspect 19. The formulation of aspect 18, comprising no greater than about 1% by weight of a sodium acrylate and sodium acryloyldimethyl taurate copolymer.

Aspect 20. The formulation of aspect 18 or 19, comprising from about 0.1% by weight to about 1.5% by weight of a sodium acrylate and sodium acryloyldimethyl taurate copolymer.

Aspect 21. The formulation of any one of aspects 3 to 20, comprising no greater than about 0.005% by weight of palmitic acid.

Aspect 22. The formulation of any one of aspects 3 to 21, comprising: from about 22% by weight to about 28% by weight 1,3-butanediol; from about 8% by weight to about 12% by weight glycerin; and from about 3% by weight to about 8% by weight 1,2-propanediol.

Aspect 23. The formulation of any one of aspects 3 to 22, comprising from about 50% by weight to about 65% by weight water.

Aspect 24. The formulation of any one of aspects 3 to 23, comprising from about 0.0005% by weight to about 0.005% by weight sphingosine.

Aspect 25. A method comprising: applying a formulation comprising at least about 50% by weight water and no greater than about 1% by weight of a sphingolipid to a region of skin of a subject to cause a sphingosine-1-phosphate biochemical pathway to be activated to produce a subsequent amount of a ceramide on the region of the skin of the subject that is greater than a baseline initial amount of the ceramide present on the region of the skin in an absence of the formulation.

Aspect 26. The method of aspect 25, wherein the subsequent amount of the ceramide is present on the region of the skin of the subject for at least 10 hours after the formulation is applied to the region of the skin of the subject.

Aspect 27. The method of aspect 25 or 26, wherein the subsequent amount of the ceramide is present on the region of the skin of the subject from about 0.5 hours to about 24 hours after the formulation is applied to the region of the skin of the subject.

Aspect 28. The method of any one of aspects 25 to 27, wherein the formulation modifies a condition of the region of the skin of the subject after being applied to the region of the skin of the subject at least once within individual consecutive 48-hour periods for at least 3 consecutive 48-hour periods.

Aspect 29. The method of aspect 28, wherein the formulation modifies a condition of the region of the skin of the subject after being applied to the region of the skin of the subject at least once per day for at least 4 consecutive days.

Aspect 30. The method of aspect 28 or 29, wherein the condition comprises at least one of dermatitis, erythema, precancerous lesions, mottling, scaling, dark spots, scars, redness, lines, or wrinkles.

Aspect 31. The method of any one of aspects 25 to 30, wherein the subsequent amount of the ceramide produced is at least about 5% greater than the baseline amount of the ceramide.

Aspect 32. The method of aspect 31, wherein the subsequent amount of the ceramide produced is at least about 10% greater than the baseline amount of the ceramide.

Aspect 33. A method of treating a skin condition comprising: applying an effective amount of a formulation comprising at least about 50% by weight water and no greater than about 1% by weight of a sphingolipid to a region of skin of a subject.

Aspect 34. The method of aspect 33, wherein the skin condition is atopic dermatitis.

Aspect 35. The method of any one of aspects 33-34, wherein the skin condition is melanoma.

Aspect 36. The method of any one of aspects 33-35, wherein the skin condition is redness.

Aspect 37. The method of any one of aspects 33-36, wherein the skin condition is dryness.

Aspect 38. The method of any one of aspects 33-37, wherein the skin condition is scarring.

Aspect 39. The method of any one of aspects 33-38, wherein the skin condition is a precancerous lesion.

Aspect 40. The method of any one of aspects 33-39, wherein the skin condition is acne.

Aspect 41. The method of any one of aspects 33-40, wherein the skin condition is a wound.

Aspect 42. The method of any one of aspects 33-41, wherein the skin condition is wrinkles.

Aspect 43. The method of any one of aspects 33-42, wherein the skin condition is hyperpigmentation.

Aspect 44. The method of any one of aspects 33-43, wherein the skin condition is dark spots.

Aspect 45. The method of any one of aspects 33-44, wherein the skin condition is erythema.

Aspect 46. The method of any one of aspects 33-45, wherein the skin condition is unevenness of skin color.

Aspect 47. The method of any one of aspects 33-46, wherein the skin condition is flakiness.

Aspect 48. The method of any one of aspects 33-47, wherein the skin condition is scaliness.

Aspect 49. The method of any one of aspects 33-48, wherein the skin condition is the presence of comedones.

Aspect 50. The method of any one of aspects 33-49, wherein application of the formulation to the region of the skin increases moisture of the skin included in the region.

Aspect 51. The method of any one of aspects 33-50, wherein application of the formulation to the region reduces redness of the skin included in the region.

Aspect 52. The method of any one of aspects 33-51, wherein application of the formulation to the region decreases inflammation of the skin included in the region.

Aspect 53. The method of any one of aspects 33-52, wherein application of the formulation to the region decreases redness of the skin included in the region.

Aspect 54. The method of any one of aspects 33-53, wherein application of the formulation of the region decreases sensitivity of the skin included in the region.

Aspect 55. The method of any one of aspects 33-54, wherein the region includes at least a portion of a face of the subject.

Aspect 56. The method of any one of aspects 33-55, wherein the region includes at least a portion of a neck of the subject.

Aspect 57. The method of any one of aspects 33-56, wherein the region includes at least a portion of at least one of a limb or appendage of the subject.

Aspect 58. The method of any one of aspects 33-57, wherein the region includes at least a portion of at least one of a torso, back, or stomach of the subject.

Aspect 59. The method of any one of aspects 33-58, wherein the region includes at least a portion of a scalp of the subject.

Aspect 60. The method of any one of aspects 33-59, wherein from 0.15 mL to 0.50 mL of the formulation is applied to the region.

Aspect 61. The method of any one of aspects 33-60, wherein the formulation is applied to the region for at least three days.

Aspect 62. The method of any one of aspects 33-61, wherein the formulation is applied to the region two times per day for at least three days.

### EXPERIMENTAL EXAMPLES

### Example 1

In a first study, eleven subjects were provided with a protocol to apply a formulation according to implementations described herein to areas of skin that were to be treated. The amount of the formulation applied per application was estimated to be from 0.15 milliliters (mL) to 0.50 mL. The subjects applied the formulation to the areas to be treated for two weeks.

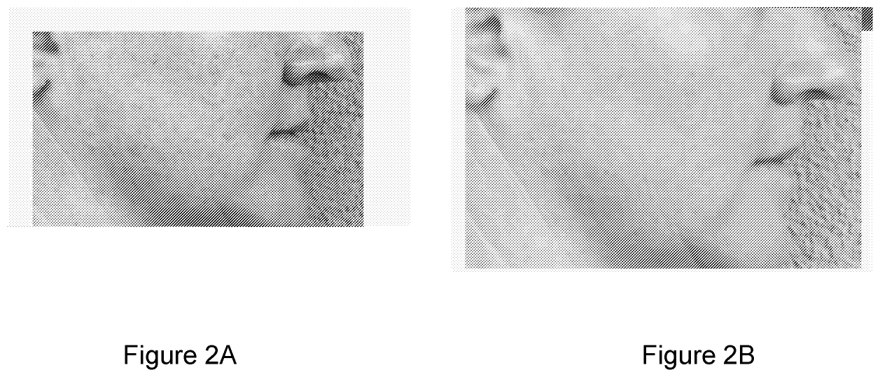

FIG. 2A shows a picture of a first subject that participated in the study prior to use of a formulation described herein and FIG. 2B shows a picture of the first subject after use of the formulation. Improvement to dermatitis was achieved in the subject. Additionally, mottling and scaling in the subject were reduced. Erythema and wrinkles were also reduced, as well as a decrease in hyperpigmentation, reduction in dark spots, and more evenness of skin tone.

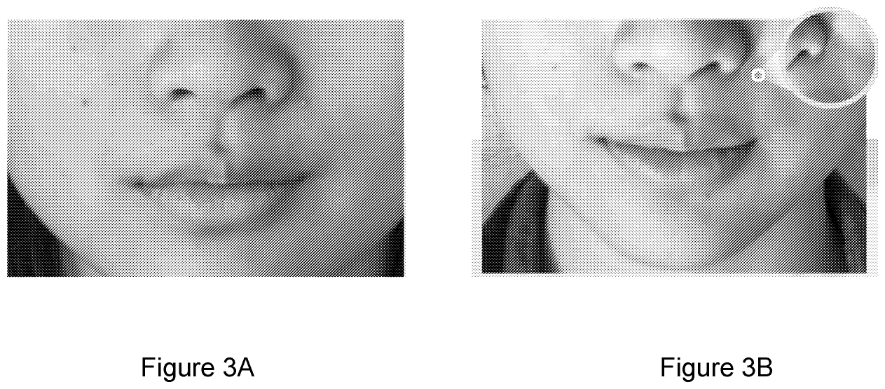

FIG. 3A shows a picture of a second subject that participated in the study prior to use of a formulation described herein and FIG. 3B shows a picture of the second subject after use of the formulation. A reduction in pimples was observed in the second subject after applying a formulation described herein.

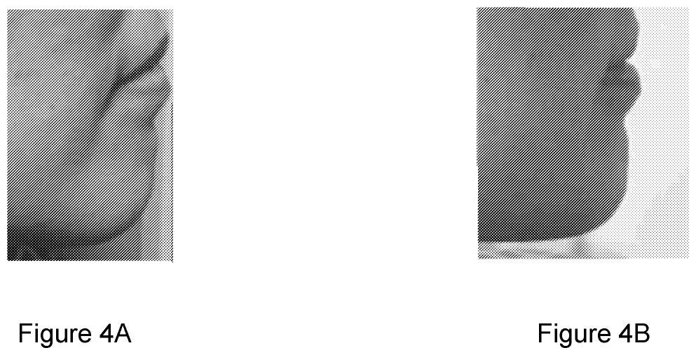

FIG. 4A shows a picture of a third subject that participated in the study prior to use of a formulation described herein and FIG. 4B shows a picture of the third subject after use of the formulation. A decrease in wrinkles including a decrease in marionette lines and a decrease in upper lip creases. The third subject also experienced a decrease in erythema and in wrinkles around the eyes.

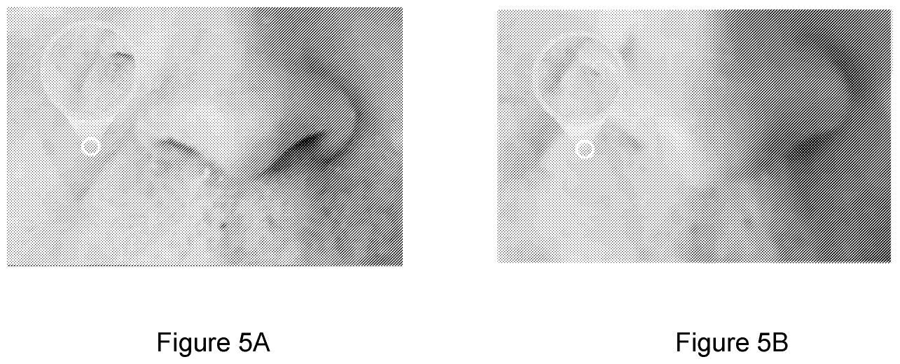

FIG. 5A shows a picture of a fourth subject that participated in the study prior to use of a formulation described herein and FIG. 5B shows a picture of the fourth subject after use of the formulation. A reduction in scaliness and flakiness was observed in the subject. Additionally, decreased mottling and decreased marionette lines were also achieved.

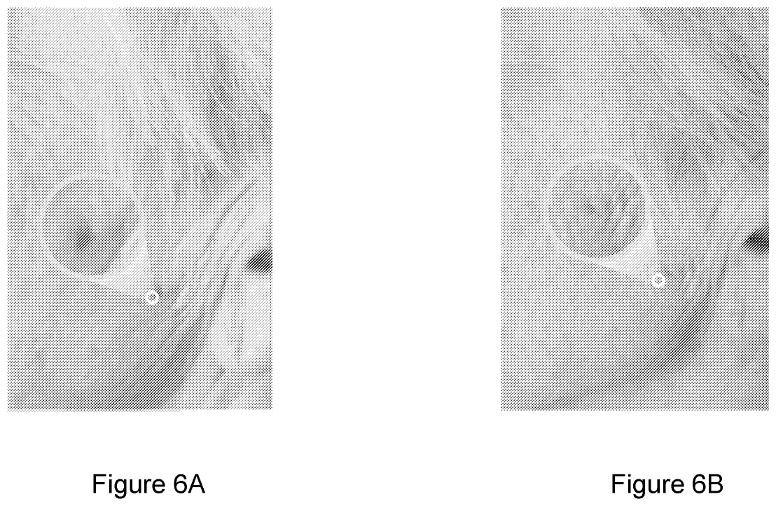

FIG. 6A shows a picture of a fifth subject that participated in the study prior to use of a formulation described herein and FIG. 6B shows a picture of the fifth subject after use of the formulation. A reduction in a precancerous lesion was observed in the fourth subject after applying a formulation described herein after 3 days of use.

### Example 2

In a second study, a total of 52 male and female subjects, ranging in age from 18 to 74 years who met all the inclusion criteria and none of the exclusion criteria as outlined in the study protocol, were selected for study participation. Recruitment was broad and based on interest in “natural skin products”

Overview of the study:
24 hr patch test before beginning topical product application
6 self assessed surveys including claims question with each item scored from 0-100
   Surveys also included open ended questions and multiple choice
Microbiome and metabolome skin swabs at beginning and end of study
Before, middle, and after photos with left, right, and straight on views
Dermal assessments of before, middle, and after photos (each with 3 views) were competed by and MD board certified dermatologist. To assess:
   1) A usable photo
   2) That the facial skin free of any serious medically treatable (ie non-cosmetic) conditions that would make assessments in this study difficult
   3) forehead lines
   4) globular folds
   5) skin around the eyes
   6) severity of upper lip creases
   7) marionette lines
   8) severity of the corner of the mouth lines
   9) severity of mottled pigmentation
   10) overall skin tone, skin health and skin vitality
   11) Glogau Wrinkle Scale
   12) Erythema (redness)
   13) Estimate the age
   14) Any additional notable changes

Participants in the study were provided with 60 mL of a topical cosmetic formulation according to implementations herein in an airless component that was used to pump a dose of the formulation. Each actuation of the pump device provided from 15 mL to 30 mL of the formulation. Participants in the study were provided with a protocol to apply the formulation using 1-2 pump actuations twice per day, once in the morning and once in the evening/night. The participants were asked to apply the doses of the formulation to the face and the neck. The study length was 15 weeks.

Statistical tests were performed as paired tests (comparing each individual's self-evaluated scores to their own previous scores. All variables were checked for normality, and where applicable Wilcoxson paired tests were used.

Table 1 shows a comparison of beginning to midpoint survey self-assessed scores (1 week compared to ˜5-6 weeks) from survey 1 to survey 3 for all participants.

| | Avg increase/ | Paired Test | |
|---|---|---|---|
| Survey question | reduction | P-value | N* |
| Moisture | +20% | <0.0001 | 41 |
| Skin Tone Evenness | +15.4% | <0.0001 | 42 |
| Itchiness | −20.4% | <0.00001 | 40 |
| Redness | −24.3% | <0.000001 | 42 |
| Sensitivity | −32.3% | <0.000000001 | 42 |
| Inflammation | −25.4% | <0.0000001 | 41 |
| Irritation | −25.2% | <0.000001 | 42 |
| Hyperpigmentation | −5.4% | <0.05 | 41 |
| Wrinkles | −19.7% | <0.000001 | 42 |
| Acne | 1.6% | NS | 41 |

N* number of participants for which paired data is available

Table 2 shows a comparison of beginning to midpoint survey self-assessed scores (week 1 compared to ˜5-6 weeks) survey 1 to survey 3 for sensitive skin participants only.

| | Avg increase/ | Paired Test | |
|---|---|---|---|
| Survey question | reduction | P-value | N* |
| Moisture | +23.2% | <0.01 | 18 |
| Skin Tone Evenness | +18.4% | <0.01 | 18 |
| Itchiness | −24.3% | <0.01 | 16 |
| Redness | −33.6% | <0.00001 | 18 |
| Sensitivity | −33.3% | <0.00001 | 18 |
| Inflammation | −31.8% | <0.001 | 17 |
| Irritation | −33.6% | <0.001 | 18 |
| Hyperpigmentation | −0.8% | NS | 18 |
| Wrinkles | −18.9% | <0.001 | 18 |
| Acne | −8.3% | NS | 17 |

Table 3 shows a comparison of beginning to midpoint survey self-assessed scores (1 week compared to ˜5-6 weeks) for non-sensitive skin participants.

| | Avg increase/ | Paired Test | |
|---|---|---|---|
| Survey question | reduction | P-value | N* |
| Moisture | +17.5 | <0.01 | 23 |
| Skin Tone Eveness | +13.9 | <0.01 | 24 |
| Itchiness | −17.8% | <0.0001 | 24 |
| Redness | −17.3 | <0.001 | 24 |
| Sensitivity | −31.5 | <0.000001 | 24 |
| Inflammation | −20.8% | <0.0001 | 23 |
| Irritation | −18.8% | <0.0001 | 24 |
| Hyperpigmentation | −0.8% | NS | 24 |
| Wrinkles | −20.4% | <0.0001 | 24 |
| Acne | −3.5% | NS | 23 |

Table 4 shows a comparison of beginning to endpoint (˜10-12 weeks) survey self-assessed scores (1 week compared to ˜10-12 weeks) for all participants.

| | Avg increase/ | Paired Test | |
|---|---|---|---|
| Survey question | reduction | P-value | N* |
| Moisture | +23.3% | <0.00001 | 43 |
| Skin Tone Evenness | +11.4% | <0.01 | 43 |
| Itchiness | −20% | <0.000001 | 41 |
| Redness | −27.3% | <0.000001 | 43 |
| Sensitivity | −37.2% | <0.00000001 | 42 |
| Inflammation | −24.2% | <0.000001 | 43 |
| Irritation | −22.8% | <0.000001 | 43 |
| Hyperpigmentation | −12.5% | <0.001 | 42 |
| Wrinkles | −20.3% | <0.0001 | 36 |
| Acne | −2.2% | NS | 42 |

Table 5 shows a comparison beginning to endpoint (˜10-12 weeks) Survey self-assessed scores (1 week compared to ˜10-12 weeks) for participants with sensitive skin.

| | Avg increase/ | Paired Test | |
|---|---|---|---|
| Survey question | reduction | P-value | N* |
| Moisture | +22.9% | <0.0001 | 19 |
| Skin Tone Evenness | +17.4% | <0.001 | 18 |
| Itchiness | −24.3% | <0.01 | 18 |
| Redness | −36.8% | <0.001 | 19 |
| Sensitivity | −45.2% | <0.000001 | 19 |
| Inflammation | −31.7% | <0.001 | 19 |
| Irritation | −30.5% | <0.001 | 19 |
| Hyperpigmentation | −10.9% | <0.01 | 18 |
| Winkles | −16.3% | <0.01 | 17 |
| Acne | −8.4% | NS | 19 |

Table 6 shows a comparison of beginning to endpoint (˜10-12 weeks) survey self-assessed scores (1 week compared to ˜10-12 weeks) for non-sensitive skin participants.

| | Avg increase/ | Paired Test | |
|---|---|---|---|
| Survey question | reduction | P-value | N* |
| Moisture | +23.4% | <0.001 | 24 |
| Skin Tone Evenness | +16.6% | <0.01 | 24 |
| Itchiness | −16.7% | <0.0001 | 23 |
| Redness | −19.9% | <0.0001 | 24 |
| Sensitivity | −30.6% | <0.001 | 23 |
| Inflammation | −18.2% | <0.001 | 24 |
| Irritation | −16.8% | <0.001 | 24 |
| Hyperpigmentation | −13.8% | <0.01 | 24 |
| Wrinkles | −23.9% | <0.001 | 19 |
| Acne | −2.9% | NS | 23 |

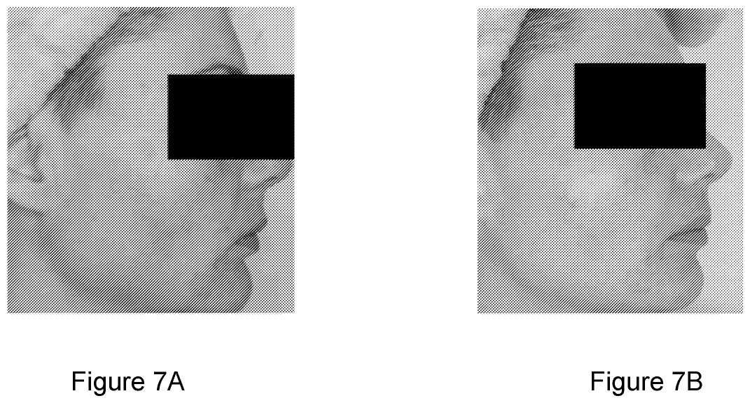

FIG. 7A shows a picture of a first subject that participated in study #2 prior to use of a formulation described herein and FIG. 7B shows a picture of the first subject after use of the formulation. The first subject experienced reduction in hyperpigmentation after use of a formulation described herein.

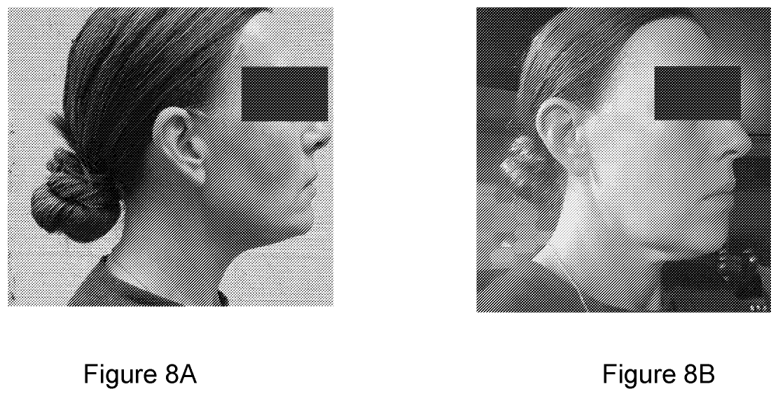

FIG. 8A shows a picture of a second subject that participated in study #2 prior to use of a formulation described herein and FIG. 8B shows a picture of the second subject after use of the formulation. A reduction in mottling of skin, redness, and hyperpigmentation were observed in the second subject after use of a formulation described herein.

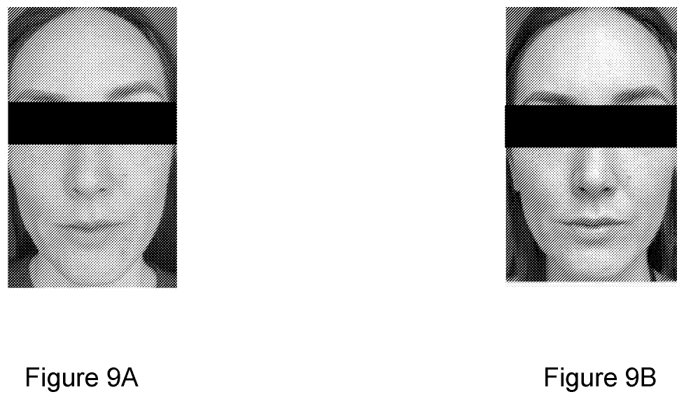

FIG. 9A shows a picture of a third subject that participated in study #2 prior to use of a formulation described herein and FIG. 9B shows a picture of the third subject after use of the formulation. The third subject experienced a reduction in hyperpigmentation, redness, and acne. Additionally, increased skin vitality was observed.

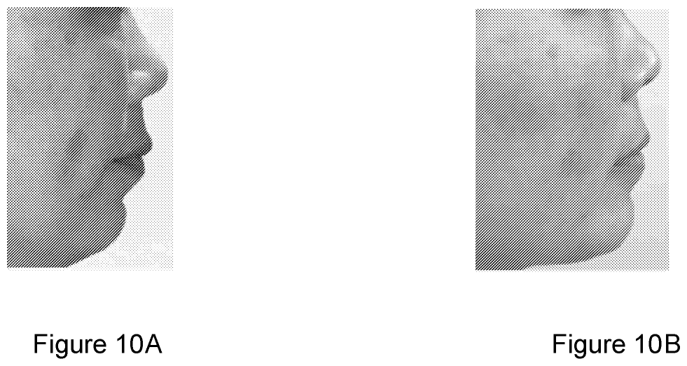

FIG. 10A shows a picture of a fourth subject that participated in study #2 prior to use of a formulation described herein and FIG. 10B shows a picture of the fourth subject after use of the formulation. A reduction in mottling and redness were observed in the fourth subject as well as improvement of the appearance of scars.

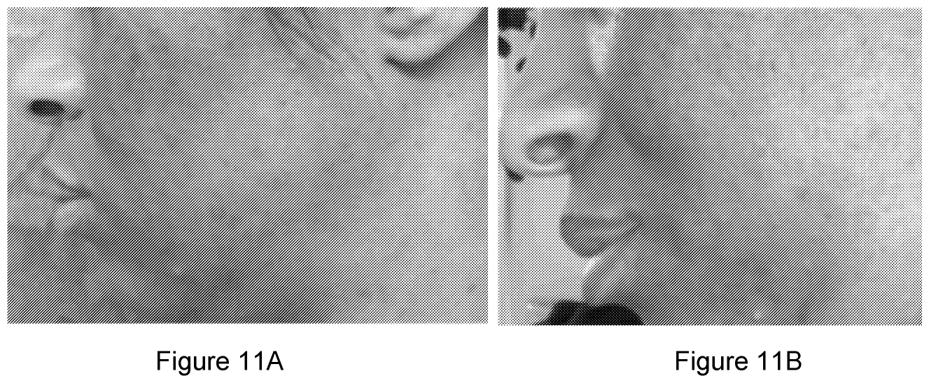

FIG. 11A shows a picture of a fifth subject that participated in study #2 prior to use of a formulation described herein and FIG. 11B shows a picture of the fifth subject after use of the formulation. A reduction in hyperpigmentation and comedones were observed in the fifth subject as well as increased vitality.

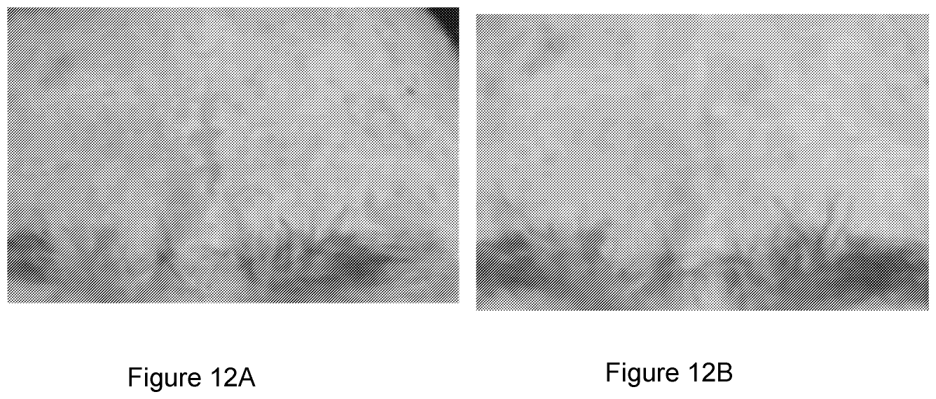

FIG. 12A shows a picture of a sixth subject that participated in study #2 prior to use of a formulation described herein and FIG. 12B shows a picture of the sixth subject after use of the formulation. A reduction in the size and prominence of scar tissue were observed in the sixth subject.

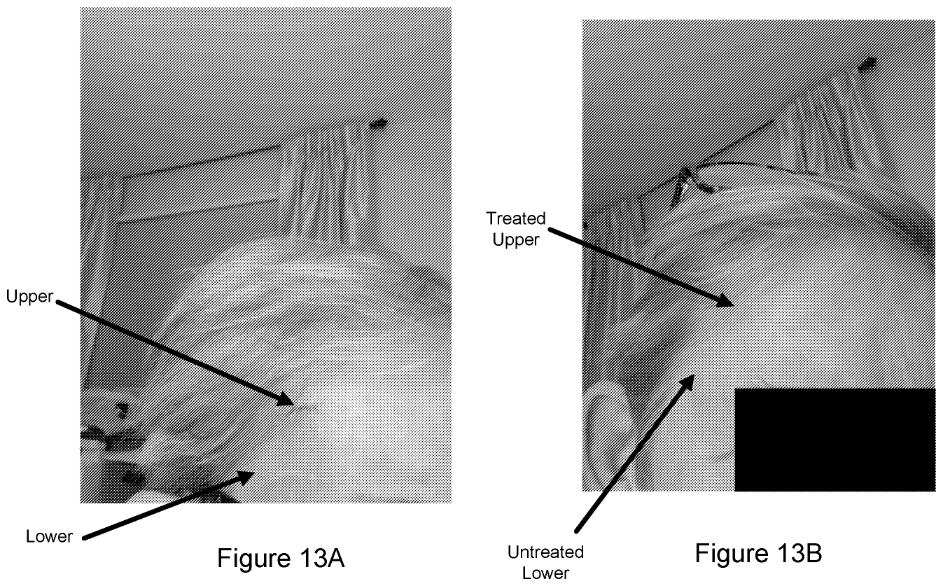

FIG. 13A shows a picture of an additional subject having an upper wound and a lower scar prior to use of a formulation described herein and FIG. 13B shows a picture of the additional subject after use of the formulation. Wound healing and lack of scarring were observed were observed in the additional subject.

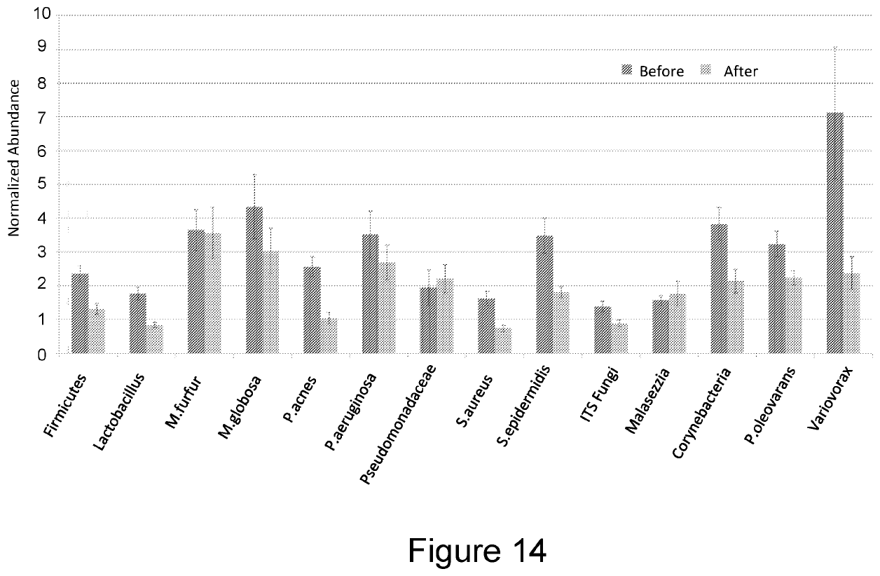

FIG. 14 shows normalized measures of abundance of a number of bacteria for subjects of study #2 characterized with normal skin before and after treatment with the formulation. FIG. 14 shows changes in the abundance of the bacteria. A more balanced skin microbiome is achieved after treatment with the formulation indicated by the relative amounts of the bacteria being closer to each other than before treatment with the formulation.

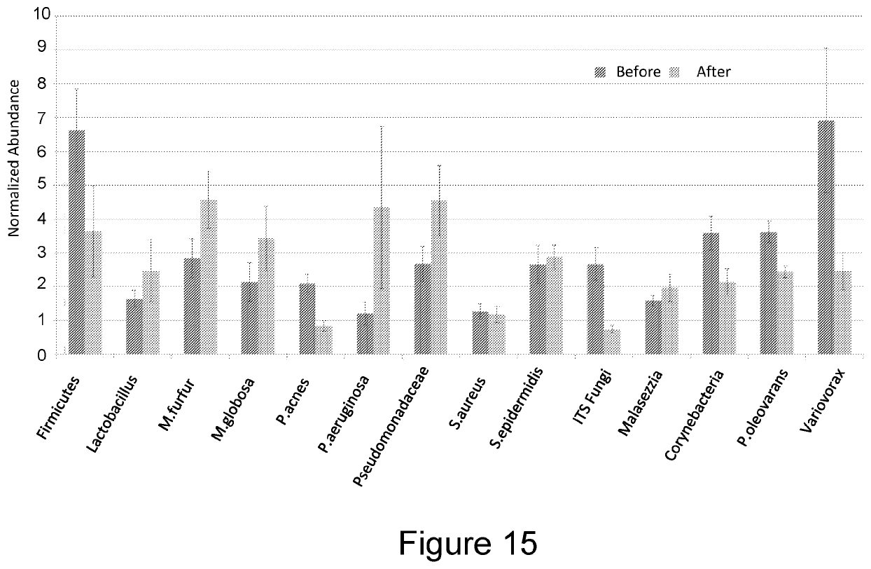

FIG. 15 shows normalized measures of abundance of a number of bacteria for subjects of study #2 characterized with sensitive skin before and after treatment with the formulation. FIG. 15 shows changes in the abundance of the bacteria. The differences between the normalized abundances of the bacteria in FIGS. 14 and 15 indicates differences in the skin microbiomes for individuals characterized with normal skin in relation to individuals characterized with sensitive skin.

The abundances of the bacteria shown in FIGS. 14 and 15 were determined according to the following procedures.

To assess effects on the skin microbiome, subjects were be provided with 2 15 mL conical tubes, each tube contains 2 pre-moistened swabs to be used to take samples from 3 face sites: 1) forehead and cheek, and 2) sides of the nose. One set of swabs was be taken from the left side of the face and the other set of swabs was be taken from the right side of the face. Sampling was performed at 1 in×1 in areas for approximately 10 seconds, with pre-moistened swabs at each site in 50:50 ethanol/water for Mass Spec (MS) analysis or 50 mM Tris pH 7.6, 1 mM EDTA, and 0.5% Tween 20 for nucleic acid analysis.

The swabs were taken when subjects woke up in the morning before washing their face or applying anything to their face. These samples were taken at the beginning of the study before the course of the formulation is started and used, and also at the end of the study.

After collection, one pair of swabs/tube was extracted in 500 μL of 50:50 ethanol/water (for mass spectrometric analysis) or Tris-EDTA buffer (50 mM Tris pH 7.6, 1 mM EDTA, and 0.5% Tween 20) for bacterial DNA extraction (the other 2 replicate swabs/tube). Samples were then stored at −80° C.

The first pair or tube of swabs extracted with ethanol/water extract was submitted to mass spectrometric analysis including MALDI-TOF for metabolite, peptide, and protein detection and UPLC-QTOF for detection of smaller molecules, including tandem MS (MS/MS) of molecules for molecular network analysis.

The second set of swabs (2 swab replicates) collected were subjected to metagenome shotgun sequencing to identify the microbiome material present in the same location. 2 tubes of swabs (each tube with 2 swabs in it) were collected at the beginning and the end of the study.

Note that not all of the activities described above in the general description are required, that a portion of a specific activity may not be required, and that one or more further activities may be performed in addition to those described. Still further, the order in which activities are listed is not necessarily the order in which they are performed.

Certain features that are, for clarity, described herein in the context of separate implementations, may also be provided in combination in a single implementation. Conversely, various features that are, for brevity, described in the context of a single implementation, may also be provided separately or in any subcombination. Further, reference to values stated in ranges includes each and every value within that range.

Benefits, other advantages, and solutions to problems have been described above with regard to specific implementations. However, the benefits, advantages, solutions to problems, and any feature(s) that may cause any benefit, advantage, or solution to occur or become more pronounced are not to be construed as a critical, required, or essential feature of any or all the claims.

The specification and illustrations of the implementations described herein are intended to provide a general understanding of the structure of the various embodiments. The specification and illustrations are not intended to serve as an exhaustive and comprehensive description of all of the elements and features of apparatus and systems that use the structures or methods described herein. Separate implementations may also be provided in combination in a single implementation, and conversely, various features that are, for brevity, described in the context of a single implementation, may also be provided separately or in any subcombination. Further, reference to values stated in ranges includes each and every value within that range. Many other implementations may be apparent to skilled artisans only after reading this specification. Other implementations may be used and derived from the disclosure, such that a structural substitution, logical substitution, or another change may be made without departing from the scope of the disclosure. Accordingly, the disclosure is to be regarded as illustrative rather than restrictive.

1. A formulation comprising:
at least about 50% by weight water;
palmitic acid; and
no greater than about 1% by weight of sphingosine.

2. (canceled)

3. The formulation of claim 1, comprising from about 20% by weight to about 40% by weight of one or more diols having from 3 to 6 carbon atoms.

4. The formulation of claim 3, comprising from about 20% by weight to about 40% by weight of a first diol having from 3 to 6 carbon atoms and from about 2% by weight to about 10% by weight of a second diol having from about 3 to 6 carbon atoms.

5. The formulation of claim 4, comprising from about 20% by weight to about 30% by weight of 1,3-butanediol and from about 2% by weight to about 10% by weight of 1,2-propanediol.

6. The formulation of claim 1, comprising from about 7% by weight to about 12% by weight glycerin.

7. The formulation of claim 1, comprising one or more conditioning agents, wherein the one or more conditioning agents include at least one of 1.3 butanediol, isohexadecane, or squalene.

8. (canceled)

9. (canceled)

10. The formulation of claim 1, comprising from about 0.10% by weight to about 0.40% by weight isohexadecane.

11. (canceled)

12. The formulation of claim 7, comprising from about 0.7% by weight to about 1.2% by weight squalene.

13. The formulation of claim 1, comprising an emulsifying agent.

14. (canceled)

15. The formulation of claim 13, comprising from about 0.06% by weight to about 0.15% by weight polysorbate 80.

16. (canceled)

17. (canceled)

18. The formulation of claim 1, comprising from about 0.1% by weight to about 1.5% by weight of a sodium acrylate and sodium acryloyldimethyl taurate copolymer.

19. (canceled)

20. The formulation of claim 1, comprising:
from about 22% by weight to about 28% by weight 1,3-butanediol;
from about 8% by weight to about 12% by weight glycerin; and
from about 3% by weight to about 8% by weight 1,2-propanediol.

21. The formulation of claim 1, comprising from about 50% by weight to about 65% by weight water.

22. The formulation of claim 1, comprising from about 0.0005% by weight to about 0.005% by weight sphingosine.

23. A formulation comprising:
from about 50% by weight to about 65% by weight water;
from about 20% by weight to about 30% by weight 1,3-butanediol;
from about 5% by weight to about 15% by weight glycerin;
from about 2% by weight to about 10% by weight 1,2-propanediol; and
from about 0.0005% by weight to about 0.005% by weight sphingosine.

24. The formulation of claim 1, comprising:
from about 0.7% by weight squalene to about 1.2% by weight squalene and
from about 0.0015% by weight palmitic acid to about 0.0025% by weight palmitic acid.

25. A method comprising:
applying a formulation comprising at least about 50% by weight water, palmitic acid, and no greater than about 1% by weight of sphingosine to a region of skin of a subject to cause a ceramide biochemical pathway to be activated to produce a subsequent amount of a ceramide on the region of the skin of the subject that is greater than a baseline initial amount of the ceramide present on the region of the skin in an absence of the formulation.

26. (canceled)

27. (canceled)

28. The method of claim 25, wherein the formulation modifies a condition of the region of the skin of the subject after being applied to the region of the skin of the subject at least once within individual consecutive 48-hour periods for at least 3 consecutive 48-hour periods.

29. (canceled)

30. The method of claim 28, wherein the condition comprises at least one of dermatitis, erythema, precancerous lesions, inflammation, mottling, scaling, dark spots, scars, redness, lines, or wrinkles.

31.-61. (canceled)

62. The formulation of claim 1 comprising, no greater than about 1% by weight palmitic acid.
